(19) United States
(12) Patent Application Publication
(10) Pub. No.: US 2007/0264367 A1
(43) Pub. Date: Nov. 15, 2007

(54) Composition containing an extract of shisandrae fructus for preventing and treating metabolic bone diseases, oxidative stress-induced diseases and inflammatory diseases

(76) Inventor: Sung-Jin Kim, Seoul (KR)

Correspondence Address: STETINA BRUNDA GARRED & BRUCKER, 75 ENTERPRISE, SUITE 250, ALISO VIEJO, CA, 92656, US

(21) Appl. No.: 11/648,326

(22) Filed: Dec. 29, 2006

(30) Foreign Application Priority Data
May 9, 2006 (KR) 10-2006-41412

(51) Int. Cl. A61K 36/79; A61P 39/00

(52) U.S. Cl. 424/769

(57) ABSTRACT

Disclosed is a pharmaceutical composition containing an extract of Schisandrae Fructus for preventing and treating metabolic bone diseases, oxidative stress-induced diseases and inflammatory diseases.

The composition of the present invention contains the extract of Schisandrae Fructus showing increasing action on bone mineralization and protein biosynthesis, antioxidant action and anti-inflammation and analgesic actions, and can be used as a direct therapeutic agent or a therapeutic aide for metabolic bone diseases, oxidative stress-induced diseases and inflammatory diseases.

A therapeutic agent of the present invention is of a natural medicament containing the extract of Schisandrae Fructus, that is, a kind of medicinal plants, and shows the above described actions without any adverse side effect shown in conventional synthetic medicaments, thereby preferably replacing the conventional synthetic medicaments.

### CROSS-REFERENCE TO RELATED APPLICATIONS

This application is based on and claims priority to Korean Patent Application No. 10-2006-41412 filed on May 9, 2006 in the Korean Intellectual Property Office (KIPO), the entire contents of which are hereby incorporated by reference.

### STATEMENT RE: FEDERALLY SPONSORED RESEARCH/DEVELOPMENT

Not Applicable

### BACKGROUND

1. Field of the Invention

The present invention relates to a pharmaceutical composition containing an extract of Schisandrae Fructus for preventing and treating metabolic bone diseases, oxidative stress-induced diseases and inflammatory diseases, more particularly to a pharmaceutical composition that can contain an extract of Schisandrae Fructus showing increasing action on bone mineralization and protein biosynthesis, antioxidant action and anti-inflammation and analgesic actions, and that can be used as a direct therapeutic agent or as a therapeutic aid for preventing and treating bone diseases, oxidative stress-induced diseases and inflammatory diseases.

2. Description of the Related Art

Metabolic bone diseases include osteoporosis, osteomalacia, osteopenia, hypophosphatemia, rickets, renal osteodystrophy, bone loss in oral and maxillofacial area, periodontal diseases, and others.

Osteoporosis, which is a representative metabolic bone disease, induced by low bone mass and dramatically decreases the quality of life. Osteoporosis shows relatively high mortality rate, and it is reported that 20% of patients suffering from hip fracture due to osteoporosis have died within one year [NIH Consensus development Panel on Osteoporosis Prevention, Diagnosis and Therapy. Osteoporosis, prevention, diagnosis, and therapy. JAMA. 2001,;285:785-795]. Currently, the number of patients with osteoporosis reaches 5,000,000 to 8,000,000 in the United States, and approximately 1,300,000 patients among them are afflicted with fractures: vertebral fracture is 50% and hip fracture is 25% [Looker A C et al. J Bone Miner Res. 12, 1762-1768, 1997; Consensus development conference: diagnosis, prophylaxis, and treatment of osteoporosis. Am J Med, 94, 645-650, 1993; Lindsay R et al., JAM, 285, 320-323, 2001]. World Health Organization (WHO) estimates that the number of patients with hip fracture due to osteoporosis will reach 6,300,000 by the year 2050, world widely [Ray N F et al. J Bone Miner Res. 12, 24-35, 1997]. Risk factors leading to the onset of osteoporosis include aging, female gender, family history or personal history of osteoporosis or fracture in adults, white race, small bone structure, low body weight, premature menopause, smoking cigarettes, steroid drug administration, and others. Particularly, it is reported that the attack rate of osteoporosis is high in white women and Asian women [Consensus development conference: diagnosis, prophylaxis, and treatment of osteoporosis. Am J Med, 94, 646-650; Siris E S et al., JAMA 286, 2815-2822, 2001].

The bone tissue is an organ system supporting the body as well as body weight and allowing body motion as attaching muscle thereto, and maintains balance through continuous remodeling creating bone formation by osteoblast together with bone resorption by osteoclast. Such metabolism is regulated by physical affections, hormone systems and local factors. If bone resorption exceeds bone formation as affected by these various factors, bone mass is decreased to below a limited amount, and metabolic bone diseases, such as osteoporosis, occur.

Alkaline phosphatase participating in bone mineralization, type I collagen, osteocalcin and others are clinically used as biochemical markers of osteoblast. The alkaline phosphatase, as an early marker of osteoblast activity, is released right after completion of osteoblast proliferation, then is decreased when mineralization of bone matrix is initiated. The collagen is a kind of bone matrix proteins, and holds approximately 90% in bone organic matrix [Schonau and Rauch, Horm. Res., 49 (supp15): 50-59, 1997]. The majority thereof is type I collagen generated in the same gene to that of the skin, and a small amount of type V collagens are present therein. The collagen provides a place where inorganic salt is deposited rather than having a simple structure, and participates in differentiation and proliferation of osteoblast so as to play a significant role in bone functions, such as calcification capacity and bone remodeling and others. Further, the collagen, as an early marker of osteoblast activity, is generated during preosteoblast differentiation. Hydroxyproline holds approximately 14% in the collagen content, and such ratio is maintained relatively regularly.

Oxidative stress is present in case of an imbalance between production of reactive oxygen species (ROS), such as superoxide, peroxide and hydroxyradical of free radicals, and scavenging of ROS and reproduction of damaged complexes. The oxidative stress induces harmful reactions, such as lipid peroxidation, apoptosis and others. Oxidative stress-induced diseases include cancer, arthritis, chronic obstructive pulmonary disease, cardiovascular disease, paralysis, AIDS, Alzheimer's disease, Parkinson's disease, alcohol addiction, periodontitis, inflammatory bowel disease, colorectal disease, bladder cancer, coronary heart disease, Huntington's disease, chronic kidney disease, alcoholic liver disease, insulin resistance syndrome, diabetes and others [Valko M et al., Chem Biol Interact, 160, 1-40, 2006; McMord J M, Am J Med, 10, 652-659, 2000; Wei Pf et al., J Periodont Res, 39, 287-293, 2004; Dryden G W et al., Curr Gastroenterol Rep, 7, 308-316, 2005; Saygili El et al., Biochemistry 68, 325-328, 2003; Sonmez H et a;., Biochemistry 68, 346-348, 2003; Maxwell S R, Basic Res Cardiol, 95, 165-171, 2000; Berlongan C V et a;., 83, 335-341,1996; Martin C J, Geoddeke-Merickel C M, Nephrol Nur J, 32, 683-685, 2005; Albano E, Free radical Biology and Medicine 32, 110-114, 2002; Ceriello A, Motz E, Arterioscler Thromb Vasc Biol, 24, 816-823, 2004].

Inflammation response is an overall continuous process of nonspecific immune stimulation with inflammation or tissue damage. Such process is generally well regulated, however, over production of an inflammatory mediator, such as cytokine, induces a variety of diseases [Delves P J et al., N Eng J med 343, 108-117, 2000]. Further, over release of bradykinin activates a bradykinin receptor and thus induces pains, dropsy, loss of tissue function, and others. The Bradykinin induces release of cytokine and noncytokine inflammatory mediators, such as PGE₂, PGI₂, LTs (leukotrienens), histamine, PAF, IL-1, TNF and others from polymorphonuclear leucocyte, macorphage, endothelial cell and synovial tissue [Sharma J N, buchanan W W, Exp Toxicol Pathol, 46, 421-433, 1994; Lerner U H, Oral Surg Oral Med oral Pathol, 78, 481-493, 1994]. Further, inflammatory cytokine activates phospholipase A2, that is, a prostaglandin synthase, and suppresses 15-hydroxyprostaglandin dehydrogenase, that is, a prostaglandin metabolase, thereby increasing prostaglandin action.

The bradykinin is an initial substance causing inflammatory response, thereby inducing the production of cytokine and prostaglandin step by step, thereby damaging tissue and consequently causing various diseases. For instance, if a bradykinin receptor present in osteoblast and articular cartilage is activated, the production of prostaglandin is promoted. Accordingly, osteoclast is mobilized and activated, thereby causing bone resorption in alveolar bone and articulation [Ljunggren O, Lerner U H, Br J Pharmacol 101, 382-386, 1990; Lemer U H, Oral Surg Oral Med oral Pathol, 78, 481-493, 1994].

Over release of inflammatory mediators, such as prostaglandin, cytokine and others, due to the production of bradykinin causes inflammatory diseases including allergic disease, rhinitis, asthma, acute pains, chronic pains, periodontitis, gingivitis, inflammatory bowel disease, gout, coronary thrombosis, atherosclerosis, congestive heart failure, hypertension, angina pectoris, stomach ulcer, Alzheimer's disease, cerebral infract, Down syndrome, multiple sclerosis, obesity, diabetes, dementia, depression, schizophrenia, tuberculosis, sleep disorder, septicemia, bums, pancreatitis and others [Henneth M, J Pharm Pharmaceut Sci 6, 602-625, 2005; Sharma J N, Buchanan W W, exp Toxicol Pathol, 46, 421-433, 1994]. Accordingly, bradyknin antagonist blocks early initial step of inflammation, and is thus useful as prophylactic and therapeutic agents for a variety of inflammatory diseases.

Schisandra Fructus (Maximowicziae Fructus) is obtained by drying fruits of Schisandra Chinensis BAILLON (=Maximowiczia chinensis RUPRECHT), Schisandra Chinensis BAILLON var. glabrata NAKAI (=Maximowiczia chinensis RUPRECHT var. glabrata NAKAI), Schisandra Chinensis REHD. Et WILS., Schisandra nigra MAX. (=Maximowiczia nigra NAKAI) and Kodsura japonica DUNAL which belong to Schisandraceae. The Schisandra Fructus contains Schizandrin, deoxyschizandrin, γ-Schizandrin, gomisin A-Q, acetylgomisin P, tigloylogomisin, benzoylgomisin Q and benzoylgomisine P, wuweizi A, wuweizi B, citral, d-ylangene, citiric acid, ascorbic acid, succinic acid, malic acid, anthocyanin, α-chamigrene, β-chamigrene, β-chamigrenol, sterol, tocopherol, oleic acid, linoleic acid, stearic acid, palmitic acid, myristic acid, palmitoleic acid and others, and is used for astringents, antitussives/antibacterial agents, antihypertension agent, cardiotonic agents and tonic [Yook Chang-soo et al., Modern Biochemistry, 24-128, 1993].

However, any efficacy of Schisandra Fructus against bone metabolic diseases, oxidative stress-induced diseases and inflammatory diseases has not yet been reported.

### BRIEF SUMMARY

The present inventor found that an extract of Schisandra Fructus promotes bone matrix protein biosynthesis and osteoblast differentiation, and has antioxidant action and anti-inflammation and analgesic actions, and thus can be used for preventing and treating metabolic bone diseases, oxidative stress-induced diseases and inflammatory diseases.

Accordingly, an object of the present invention is to provide a nature-originated medicament for preventing and treating bone metabolism diseases, oxidative stress-induced diseases and inflammatory diseases that can replace conventional synthetic medicaments showing adverse side effects.

Additional advantages, objects and features of the invention will be set forth in part in the description which follows and in part will become apparent to those having ordinary skill in the art upon examination of the following or may be learned from practice of the invention.

In order to accomplish these objects, there is provided a composition containing an extract of Schisandra Fructus for preventing and treating metabolic bone diseases, oxidative stress-induced diseases and inflammatory diseases.

The extract of Schisandra Fructus may be extracted by following extraction methods of medicinal herbs.

Extraction process 1: Schisandra Fructus is dissolved in a solvent selected from the group consisting of lower alcohol having a carbon number of 1 to 4, alcohol-water mixture, lower acetate, such as ethylacetate, acetone, chloroform, dichloromethane, carbon tetra chloride, methylenechloride, ether, petroleum ether or hexane, in order to obtain soluble fractions. Herein, reaction temperature is 5 to 80° C., preferably 30 to 55□, and reaction time is 15 minutes to 48 hours, preferably 30 minutes to 12 hours.

Extraction process 2: Water is added to the residue of Schisandra Fructus extracted in the above extraction method 1, in order to obtain a water-soluble fraction. Herein reaction temperature is 5 to 80°, preferably 30 to 55°, and reaction time is 15 minutes to 48 hours, preferably 30 minutes to 12 hours.

Further, the extract of Schisandra Fructus of the present invention may be additionally subjected to following fractioning processes as conventionally involved in fractionation and separation methods through adjusting pH [Harborne J B. Phytochemical methods: A guide to modern techniques of plant analysis. 3rd Ed. Pp 6-7, 1998].

Extraction process 3: The extract of Schisandra Fructus obtained in the above extraction method is dissolved in a mixed solvent of lower alcohol and water, followed by adjusting pH 2-4 and adding the same amount of chloroform thereto, in order to obtain a chloroform-soluble fraction.

Extraction process 4: Chloroform-insoluble fractions are adjusted to pH 9-12 with ammonium hydroxide, followed by extracting and fractioning with a mixed solvent of chloroform/methanol, in order to obtain a chloroform/methanol-soluble fraction. Herein, a mixing ratio of chloroform:methanol is preferably 1:0.1-1. Alkaloids are present mostly in the chloroform/methanol-soluble fraction among the chloroform-insoluble fractions, while quaternary alkaloids and N-oxides are present in the methanol-soluble fraction among chloroform/methanol-insoluble fractions.

Extraction process 5: The chloroform/methanol-insoluble fractions are further extracted and fractioned with methanol in order to obtain a methanol-soluble fraction.

A therapeutic agent of the present invention, is as a natural medicament containing the extract of Schisandra Fructus, that is, a kind of medicinal plants, and shows excellent improving action on osteoblast functions, antioxidant action and anti-inflammation and analgesic actions without any adverse side effect shown in conventional synthetic medicaments. Therefore, the therapeutic agent may be used for preventing and treating metabolic bone diseases, oxidative stress-induced diseases and inflammatory diseases, and may preferably replace the conventional synthetic medicaments.

Recommended daily dosage of the therapeutic agent containing the extract of Schisandra Fructus for treating stress-induced diseases, inflammatory diseases and metabolic bone diseases is preferably 0.01-0.10 g/kg of body weight in the early stages of treatment. However, such dosage may be altered according to medication necessary of patients, disease states and compounds to be used.

According to the present invention, in order to clinically administer the therapeutic agent for treating stress-induced diseases, inflammatory diseases and metabolic bone diseases, a pharmaceutically acceptable inactive carrier is mixed with the extract of Schisandra Fructus, and then the mixture may be formulated into a solid, semi-solid or liquid dosage form applicable to oral or non-oral administration. The pharmaceutically acceptable inactive carrier can be either solid or liquid form, and may be one or more selected from ingredients acting as diluent, flavoring agent, solubilizing agent, lubricant, suspending agent, binding agent and disintegrant.

### BRIEF DESCRIPTION OF THE DRAWINGS

The above and other objects, features and advantages of the present invention will be more apparent from the following detailed description taken in conjunction with the accompanying drawing, in which:

FIG. 1 shows increasing effect of an extract of Schisandra Fructus on collagen biosynthesis of UMR-160 cell, that is, an osteoblast-like cell. Values, shown therein, are mean±standard deviation (n=4), and significance in the control group is defined as *: P<0.05; * *: P<0.01;

FIG. 2 shows effect of an extract of Schisandra Fructus on alkaline phosphatase activity of UMR-160 cell, that is, an osteoblast-like cell. Values, shown therein, are mean±standard deviation (n=4), and significance in the control group is defined as * *: P<0.01; * * * :P<0.001;

FIG. 3 shows antioxidant effect of an extract of Schisandra Fructus. Values, shown therein, are mean±standard deviation (n=3), and significance in the control group is defined as * *: P<0.01;

FIG. 4a and 4b show increasing effect of an extract of Schisandra Fructus on analgesic action in the acetic acid writhing test. Values, shown therein, are mean±standard deviation (n=6), and significance in the control group is defined as * :P<0.05; * *: P<0.01; * * *:P<0.001;

FIG. 5a and 5b show increasing effect of an extract of Schisandra Fructus on analgesic action in the tail-flick test. Values, shown therein, are mean±standard deviation (n=6), and significance in the control group is defined as * :P<0.05; * * * P<0.001; and

FIG. 6a and 6b show increasing effect of an extract of Schisandra Fructus on anti-inflammation action. Values, shown therein, are mean±standard deviation (n=4), and significance in the control group is defined as * :P<0.05; * *: P<0.01;

### DETAILED DESCRIPTION OF EXEMPLARY EMBODIMENTS

Hereinafter, preferred embodiments of the present invention will be described in detail with reference to the accompanying drawing. The aspects and features of the present invention and methods for achieving the aspects and features will be apparent by referring to the embodiments to be described in detail with reference to the accompanying drawings. However, the present invention is not limited to the embodiments disclosed hereinafter, but can be implemented in diverse forms. The matters defined in the description, such as the detailed construction and elements, are nothing but specific details provided to assist those of ordinary skill in the art in a comprehensive understanding of the invention, and the present invention is only defined within the scope of the appended claims. In the entire description of the present invention, the same drawing reference numerals are used for the same elements across various figures. % as described in the description and embodiment of the present invention means % by weight.

### Embodiment 1

Schisandra Fructus 500 g was finely pulverized and extracted with 5 fold amount of MeOH (2,500 ml) in an ultrasound extractor. The obtained extract was filtered with filter paper or multiple layers of gauze to obtain supernatant. After repeating the above extraction and filtration procedures 3 times, the obtained supernatant was concentrated using a rotary evaporator and freeze-dried in order to obtain a methanol extract. The remaining residue of Schisandra Fructus was extracted with H₂O 2,500 ml using a reflux cooler. After repeating the above extraction procedure 3 times, the obtained extract was concentrated using a rotary evaporator and freeze-dried in order to obtain a water extract.

### FORMULATION EXAMPLE 1

### Tablet

Extract of Schisandra Fructus of the embodiment 1, 5.0 mg

Lactose BP, 150.0 mg

Starch BP, 30.0 mg

Pregelatinized cornstarch BP, 15.0 mg

Magnesium stearate, 1.0 mg

The extract of Schisandra Fructus of the embodiment 1 was sieved, and mixed with lactose, starch and pregelatinized cornstarch. The mixture was added with purified water and then granulated into powder. The granules were dried, followed by mixing with magnesium stearate and applying pressure, in order to obtain tablets.

### FORMULATION EXAMPLE 2

### Capsule

Extract of Schisandra Fructus of the embodiment 1, 5.0 mg

Starch 1500, 100.0 mg

Magnesium stearate BP 1.0 mg

The extract of Schisandra Fructus of the embodiment 1 was mixed with pharmaceutical excipient, and the mixture was filled into a gelatin capsule, in order to obtain a capsule.

### FORMULATION EXAMPLE 3

### Syrup

Extract of Schisandra Fructus of the embodiment 1, 5.0 mg

Refined sugar, 637.5 g

Carboxyrnethylcellulose sodium, 2.9 g

Methyl paraben, 0.28 g

Prophyl paraben, 0.12 g

Ethanol, 20 ml

Refined sugar was dissolved in purified water 500 ml, first. Carboxymethylcellulose sodium was dissolved in purified water 400ml separately. The refined sugar- and carboxymethylcellulose sodium-dissolved solutions were mixed together. The mixed solution was added with methyl paraben and prophyl paraben, followed by adding ethanol and purified water sequentially to make 1,000 ml of a solution. The extract of Schisandra Fructus of the embodiment 1 was suspended therewith in order to obtain syrup.

### FORMULATION EXAMPLE 4

### Pastase (dentifrice)

Extract of Schisandra Fructus of the embodiment 1, 50.0 g

Cornstarch, 50.0 g

White petroleum, 100.0 g

The extract of Schisandra Fructus of the embodiment 1 and cornstarch were sieved, first. The mixture was added with white petroleum, followed by softening in order to obtain pastase.

### FORMULATION EXAMPLE 5

### Gargling Solution

Extract of Schisandra Fructus of the embodiment 1, 10.0 mg (based on dry weight)

Peppermint solution, 50.0 ml

Total amount of purified water added, 1,000 ml

The extract of Schisandra Fructus of the embodiment 1 was dissolved in purified water, first. Peppermint solution and purified water was added thereto to make a total of 1,000 ml of a solution, followed by filtering to obtain gargling solution.

### EXPERIMENTAL EXAMPLE 1

### Promoting Effect on Collagen Biosynthesis of Osteoblast

The following experiment was performed in order to ensure effect of the extract of Schisandra Fructus of the embodiment 1 on collagen biosynthesis of UMR-106 cell. Rat osteoblast-like UMR-106 cells (ATCC CRL-1661, Rockville, Md.) were cultured in DMEM medium containing fetal bovine serum(FBS) 10%, penicillin 100 IU/ml and streptomycin 100 μg/ml, in humidified 5% CO₂-95% air, at 37□, in a CO₂ incubator. The cells in early stages of thick growth were treated with trypsin, in order to measure the number thereof, and 1×10⁵ cells were inoculated into each well tissue culture plate (Corning, USA).

In order to eliminate other effects of substances of the FBS, the medium was exchanged to a different medium containing bovine serum albumin (BSA) 0.1% instead of FBS within 24 hours before treating the extracts of Schisandra Fructus. Experimental groups were treated with each extract of Schisandra Fructus 10 μg/ml, respectively, and then 24 hours later, the amount of collagen was measured.

In order to identify collagen biosynthesis in the cell, Sircol collagen assay kit (Biocolor Ltd., Northern Ireland) was used according to the present manual [Kim, S. J, et al., Biochem Biophys. Res. Commun., 278, 712-718, 2000].

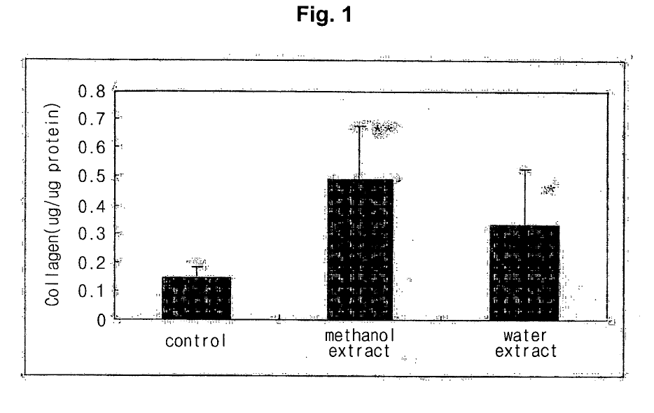

In result, when treating the cells with the methanol extract of Schisandra Fructus 10 μg/ml, the biosynthesis of collagen, that is, a major bone matrix protein was increased 3.36 folds, as compared to the control group, while the water extract thereof increased collagen biosynthesis 2.31 folds (FIG. 1).

Accordingly, the extract of Schisandra Fructus contained in the composition of the present invention as an active ingredient, increases collagen biosynthesis and thus has fixation effect on hard tissue.

### EXPERIMENTAL EXAMPLE 2

### Increasing Effect on Alkaline Phosphatase Activity

The following experiment was performed in order to ensure effect of the extract of Schisandra Fructus of the embodiment 1 on the activity of alkaline phosphatase, that is, a marker for osteoblast differentiation and bone mineralization. The alkaline phosphatase released from the culture media of osteoblasts during the culture was closely connected with bone mineralization [Sufawara Y et al., Jpn J Pharmacol, 88, 262-269, 2002].

UMR-106 cells were cultured by the same method used in the experiment example 1. The cells in early stages of thick growth were treated with trypsin, in order to measure the number thereof, and 1×10⁵ cells were inoculated into each well tissue culture plate (Corning, USA). In order to eliminate other effects of substances of the FBS, the medium was exchanged to a different medium containing bovine serum albumin (BSA) 0.1% instead of FBS within 24 hours before treating the extracts of Schisandra Fructus. Experimental groups were treated with each extract of Schisandra Fructus 10 μg/ml respectively, and then 24 hours later, the activity of alkaline phosphatase was measured. In order to identify alkaline phosphatase activity, absorbency was measured at a wavelength of 500 nm using Alkaline phosphatase assay kit (Yeong Dong bio-tech co., Ltd.).

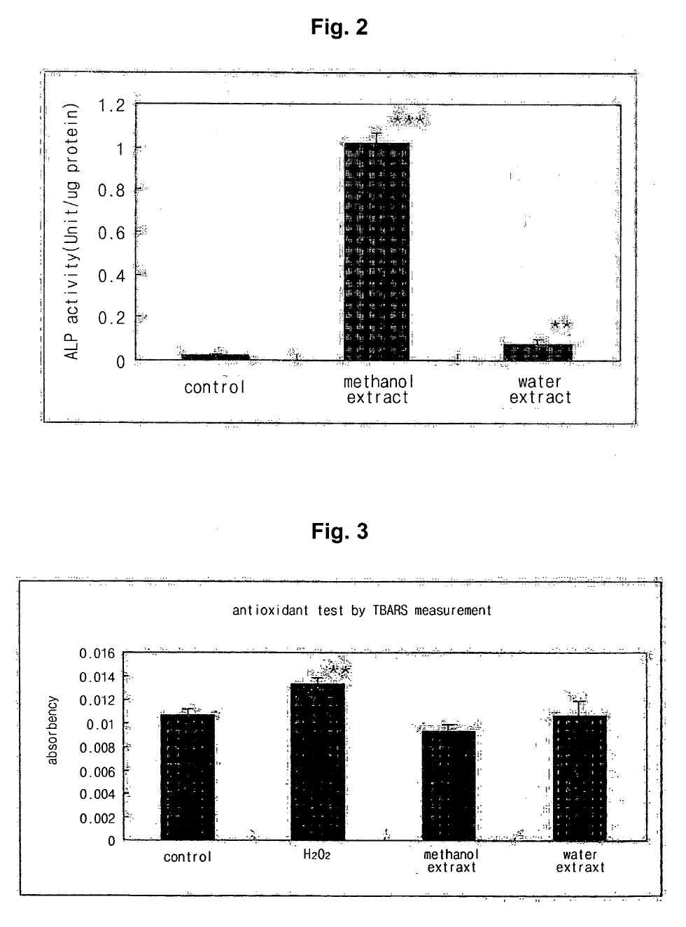

In result, when treating the cells with the methanol extract of Schisandra Fructus 10 μg/ml, alkaline phosphatase activity was increased 43.37 folds, as compared to the control group, while the water extract thereof increased alkaline phosphatase activity 3.30 folds (FIG. 2).

Accordingly, the extract of Schisandra Fructus induces an increase in alkaline phosphatase activity, thereby inducing osteoblast differentiation and consequently, influencing bone mineralization.

### EXPERIMENTAL EXAMPLE 3

### Inspection of Antioxidant Action by Measuring Lipid Peroxide (TBARS)

Reactive oxygen species (ROS), such as superoxide, peroxide and hydroxyl radical, gives rise to oxidative stress in the cell, and thus causes harmful reactions, such as lipid peroxidation, apoptosis and others. Thiobarbituric acid reactive substance (TBARS) is used as an index to measure the activity of ROS [Tuter G et al., J periodontal 72, 882-888, 2001].

UMR-106 cells were cultured by the same method used in the experiment example 1. The cells in early stages of thick growth were treated with trypsin, in order to measure the number thereof, and 1×10⁵ cells were inoculated into each well tissue culture plate (Corning, USA). In order to eliminate other effects of substances of the FBS, the medium was exchanged to a different medium containing bovine serum albumin (BSA) 0.1% instead of FBS within 24 hours before treating the extracts of Schisandra Fructus. H₂O₂ treatment group was treated with H₂O₂ (500 uM), and experimental groups were treated with the extract of Schisandra Fructus obtained in the embodiment 1 (10 μg/ml) and H₂O₂ (500 uM), respectively. 24 hours after cultivation thereof, the medium was removed, followed by rinsing with ice-cold PBS and adding an appropriate amount of Triton X-100 0.1% in order to harvest cells. The harvested cells were subjected to the reaction on ice for 30 minutes, followed by centrifuging at fill speed 10,000×g for 5 minutes in order to recover supernatant. The recovered supernatant was added with 20% acetic acid (pH 3.5) and 0.78% thiobarbituric acid in a ratio of 1:1, followed by reacting at 95□ for 1 hour. The reaction solution was cooled at room temperature, followed by centrifuging at full speed of 1,500×g for 15 minutes in order to recover supernatant. Absorbency of the recovered supernatant was measured at a wavelength of 532 nm in order to inspect antioxidant action [Cortizo A et al., Toxicology, 147, 89-99, 2000; Gutteridge J M, Hall I B, Trends Biochem Sci, 15, 129-135, 1990].

In result, when treating the cells with the methanol extract of Schisandra Fructus, the increased TBARS due to H₂O₂ was decreased 13% as compared to that of the control group, while the water extract thereof decreased TBARS to the same level with that of the control group (FIG. 3). Accordingly, the extract of Schisandra Fructus strongly inhibits the production of lipid peroxide due to oxidative stress caused by free radicals, thus can be used as an excellent antioxidant agent.

### EXPERIMENTAL EXAMPLE 4

### Measurement of Analgesic Action

### 1) Measurement of Analgesic Action Using Acetic Acid Writhing Method

Experimental and control groups comprised 6 male rats having a body weight of 20-25 g, respectively. The extract of Schisandra Fructus (10-40 mg/kg) was orally administered to the experimental group, and then after 10 minutes 0.7% acetic acid/saline solution (0.1 ml/10 g) was intraperitoneally administered thereto. Then 10 minutes later, a writhing frequency was measured for 10 minutes. Saline solution was administered to the control group instead of the extract of Schisandra Fructus, and a writhing frequency was then measured in the same way to the experimental group [Vander Wende C. and Margolian, S. Analgesic tests based upon experimentally induced acute abdominal pain in rats. Fed. Proc. 15, 494, 1956].

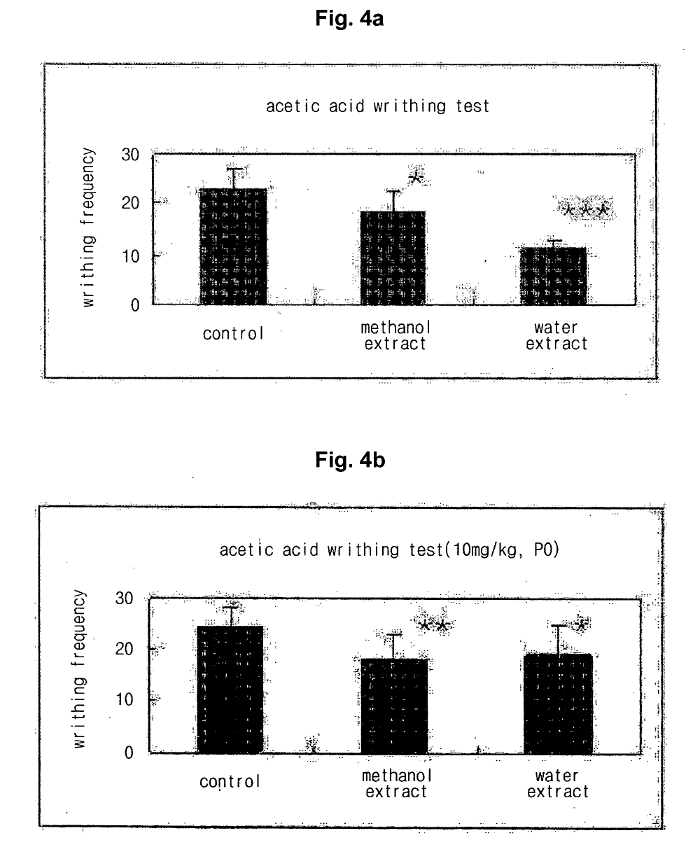

In result, when administering the extract of Schisandra Fructus 10 mg/kg, writhing occurs 18.2 times in the methanol extract treatment group and 18.8 times in the water extract treatment group, while occurring 24.3 times in the control group (FIG. 4a). Further, when administering the extract of Schisandra Fructus 40 mg/kg, writhing occurs 18.5 times in the methanol extract treatment group and 11.3 times in the water extract treatment group, while occurring 23.2 times in the control group (FIG. 4b). Thus, the extract of Schisandra Fructus decreased the writhing frequency, as compared to that of the control group.

### 2) Measurement of Analgesic Action Using Tail-Flick Method

Experimental and control groups comprised 6 male rats having a body weight of 20 g, respectively. The extract of Schisandra Fructus (10-40 mg/kg) was orally administered to the experimental group. Then 30 minutes later, infrared beam was emitted to the part 2-5 cm apart from the end of the tail of the rat using analgesimeter (Ugo basile co.), and reaction time to tail flick was then measured. Saline solution was administered to the control group instead of the extract of Schisandra Fructus, and reaction time to tail-flick was then measured in the same way to the experimental group [D'Amour, F. E., and Smith, D, L A method for determining loss of pain sensation. J. Pharmacol. Exp. Ther. 73, 74-9, 1941].

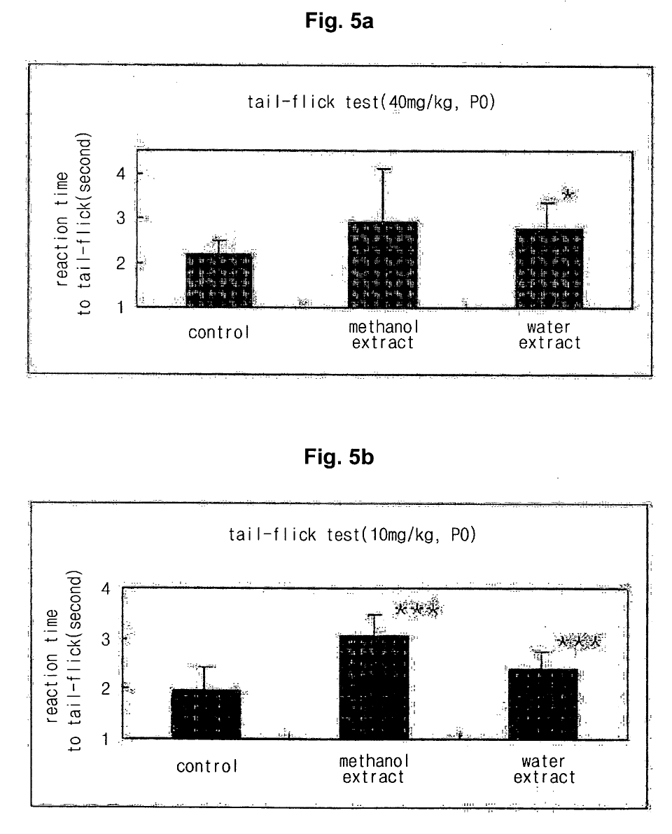

In the result, when administering the extract of Schisandra Fructus 10 mg/kg, reaction time to tail-flick was 3.10 seconds in methanol extract treatment group and 2.42 seconds in the water extract treatment group while reaction time to tail-flick of the control group was 1.97 seconds (FIG. 5a). Further, when administering the extract of Schisandra Fructus 40 mg/kg, reaction time to tail-flick was 2.93 seconds in methanol extract treatment group and 2.77 seconds in water extract treatment group, while reaction time to tail-flick of the control group was 2.18 seconds (FIG. 5a). Thus, the extract of Schisandra Fructus remarkably increased reaction time to tail-flick, as compared to that of the control group.

### EXPERIMENTAL EXAMPLE 5

### Measurement of Anti-Inflammatory Action Using Inhibitory Effect on Contraction of Ileum by Baradykinin

4 guinea pigs having a body weight of 200 g were fasted for 24 hours in order to be used for identifying how the extract of Schisandra Fructus acts on pain-inducing substances using bradykinin (50 ng/ml). Euthanasia of the guinea pigs was performed with CO₂ gas. Ileums were isolated therefrom and then cut into 1 cm long strips. The ileum strips were suspended in 10 ml organ bath (95% O₂ and 5% C O₂) at 37° C. supplying CO₂ gas thereto during the experiment. A change in tension was measured with physiograph through transducer at a resting tension of 0.5 g. Krebs-Henseleit buffer solution was used as physiological solution. The control group was placed in organ bath, followed by equilibrating under the above tension condition for 1 hour then adding bradykinin to the organ bath, in order to measure a change in tension. The experimental group was suspended in organ bath under the same condition stated above, followed by applying a tension of 5 g and equilibrating for 1 hour. The extract of Schisandrae Fructus was added to the organ bath and then 15 minutes later, bradykinin was added thereto in order to measure a change in tension. Ileum contractions were measured as a change in tension [Kim S J, Kim M S, Inhibitory effect of Cimicifugae rhizoma extracts on histamine, bradykinin and COX mediated inflammatory actions., Phototherapy Res. 14, 596-600, 2000].

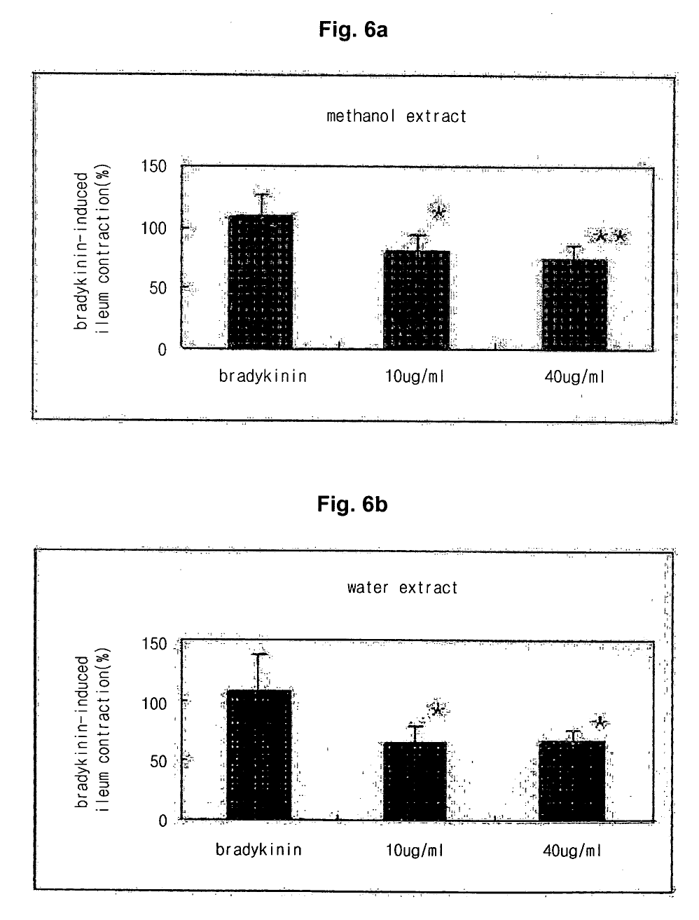

In the result, the methanol extract of Schisandra Fructus inhibited the ileum contraction by bradykinin, 28% when added at 10 ug/ml, and 35% when added at 40 ug/ml (FIG. 6a). The water extract of Schisandra Fructus inhibited the ileum contraction by bradykinin, 44% when added at 10 ug/ml, and 42% when added at 40 ug/ml (FIG. 6b).

As described above, the composition containing an extract of Schisandra Fructus for preventing and treating metabolic bone diseases, oxidative stress-induced diseases and inflammatory diseases, according to the present invention, produces the following effects.

First, the therapeutic agent according to the present invention is a natural medicament containing the extract of Schisandra Fructus, that is, a kind of medicinal plants, and shows excellent improving action on osteoblast functions, antioxidant action and anti-inflammation and analgesic actions without any adverse side effect shown in conventional synthetic medicaments.

Second, the therapeutic agent can be used for preventing and treating metabolic bone diseases, oxidative stress-induced diseases and inflammatory diseases, and can preferably replace the conventional synthetic medicaments.

It should be understood by those of ordinary skill in the art that various replacements, modifications and changes in the form and details may be made therein without departing from the spirit and scope of the present invention as defined by the following claims. Therefore, it is to be appreciated that the above described embodiment are for purposes of illustration only and are not to be construed as limitations of the invention.

What is claimed is:

1. A pharmaceutical composition containing an extract of Schisandra Fructus for preventing and treating metabolic bone diseases, oxidative stress-induced diseases and inflammatory diseases

2. The composition of claim 1, wherein the extract of Schisandra Fructus is obtained by extracting Schisandra Fructus with a solvent selected from the group consisting of lower alcohol having a carbon number of 1 to 4, alcohol-water mixture, lower acetate, such as ethylacetate, acetone, chloroform, dichloromethane, carbon tetra chloride, methylenechloride, ether, petroleum ether or hexane.

3. The composition of claim 1, wherein the extract of Schisandra Fructus is obtained by extracting the residue of Schisandra Fructus extracts, which is extracted with a solvent selected from the group consisting of lower alcohol having a carbon number of 1 to 4, alcohol-water mixture, lower acetate, such as ethylacetate, acetone, chloroform, dichloromethane, carbon tetra chloride, methylenechloride, ether, petroleum ether or hexane, with water.

4. The composition of claim 1, wherein metabolic bone diseases include osteoporosis, osteomalacia, osteopenia, hypophosphatemia, rickets, renal osteodystrophy, bone loss in oral and maxillofacial area or periodontal diseases.

5. The composition of claim 1, wherein oxidative stress-induced diseases include cancer, arthritis, chronic obstructive pulmonary disease, cardiovascular disease, paralysis, AIDS, Alzheimer's disease, Parkinson's disease, alcohol addiction, periodontitis, inflammatory bowel disease, colorectal disease, bladder cancer, coronary heart disease, Huntington's disease, chronic kidney disease, alcoholic liver disease, insulin resistance syndrome or diabetes.

6. The composition of claim 1, wherein inflammatory diseases include allergic disease, rhinitis, asthma, acute pains, chronic pains, periodontitis, gingivitis, inflammatory bowel disease, gout, coronary thrombosis, atherosclerosis, congestive heart failure, hypertension, angina pectoris, stomach ulcer, Alzheimer's disease, cerebral infarct, Down syndrome, multiple sclerosis, obesity, diabetes, dementia, depression, schizophrenia, tuberculosis, sleep disorder, septicemia, bums or pancreatitis.

7. The composition of claim 1, wherein the composition is an additive of pastase and gargling solution.

8. A dietary supplement containing the extract of Schisandra Fructus for preventing and treating metabolic bone diseases, oxidative stress-induced diseases and inflammatory diseases.
